(19) United States
(12) Plant Patent Application Publication
(10) Pub. No.: US 2003/0070194 P1
(43) Pub. Date: Apr. 10, 2003

(54) Miniature rose plant named 'Meimiento'

(76) Inventor: Alain A. Meilland, Antibes (FR)

Correspondence Address: Benton S. Duffett, Jr., BURNS, DOANE, SWECKER & MATHIS, L.L.P., P.O. Box 1404, Alexandria, VA, 22313-1404, US

(21) Appl. No.: 10/294,687

(22) Filed: Nov. 15, 2002

Related U.S. Application Data
(63) Continuation of application No. 09/420,795, filed on Oct. 19, 1999.

(51) Int. Cl. A01H 5/00

(52) U.S. Cl. PLT/121

(57) ABSTRACT

A new and distinct variety of rose plant of the Miniature Class is provided which abundantly forms attractive double light pink blossoms. The buds are elongated and commonly open slowly. The growth habit is low compact and bushy. Decorative dark green foliage is formed that contrasts nicely with the light pink blossoms. Very good disease resistance is exhibited under greenhouse growing conditions.

### SUMMARY OF THE INVENTION

The new variety of Miniature rose plant was created by artificial pollination wherein two parents were crossed which previously had been studied in the hope that they would contribute the desired characteristics. The female parent (i.e., the seed parent) of the new variety was the ‘Meichevil’ variety (U.S. Plant Pat. No. 7,334). The male parent (i.e., the pollen parent) was the product of the pollination of the ‘Meilarco’ variety (U.S. Plant Pat. No. 6,170) and the ‘Meijikatar’ variety (U.S. Plant Pat. No. 4,682). The parentage of the new variety can be summarized as follows:

‘Meichevil’×(‘Meilarco’×‘Meijikatar’

The seeds resulting from the above pollination were sown and small plants were obtained which were physically and biologically different from each other. Selective study resulted in the identification of a single plant of the new variety.

It was found that the new variety of Miniature rose plant of the present invention possesses the following combination of characteristics:

(a) exhibits a low compact and bushy growth habit,

(b) forms elongated buds that commonly open slowly,

(c) forms in abundance attractive double light pink blossoms,

(d) forms decorative dark green foliage, and

(e) exhibits very good resistance to diseases under greenhouse growing conditions.

The prolonged opening of the buds to form flowers enhances the attractiveness of the overall plant wherein the presence of the light pink buds and blossoms contrasts nicely with the dark green foliage.

The new variety well meets the needs of the horticultural industry. It is particularly well-suited for pot forcing under greenhouse growing conditions to form an attractive gift plant.

The new variety has been found to undergo asexual propagation in France by a number of routes, including budding, grafting, and cuttage. Asexual propagation by the above-mentioned methods as performed in France has shown that the characteristics of the new variety are strictly transmissible from one generation to another.

The new variety has been named the ‘Meimiento’ variety.

### BRIEF DESCRIPTION OF THE PHOTOGRAPH

The accompanying photograph shows as nearly true as it is reasonably possible to make the same, in a color illustration of this character, typical specimens of the plant parts of the new variety. The rose plants of the new variety were two years of age and were observed during March while growing on Rosa indica understock in greenhouses at LeCannet des Maures, Var, France. A page (i.e., Red Group 56) from the R.H.S. Colour Chart of The Royal Horticultural Society, London, is included for comparative purposes. Dimensions in centimeters are shown at the bottom of the photograph.

FIG. 1—illustrates a specimen of a young shoot;

FIG. 2—illustrates specimens of two floral buds before the opening of the sepals;

FIG. 3—illustrates specimens of two floral buds at the opening of the sepals;

FIG. 4—illustrates specimens of two floral buds at the opening of the petals;

FIG. 5—illustrates specimens of two flowers during the course of opening;

FIG. 6—illustrates a specimen of an open flower—plan view—obverse;

FIG. 7—illustrates a specimen of an open flower—plan view—reverse;

FIG. 8—illustrates a specimen of a fully open flower—plan view obverse;

FIG. 9—illustrates a specimen of a fully open flower—plan view—reverse;

FIG. 10—illustrates a specimen of a floral receptacle showing the arrangement of the stamens and pistils; FIG. 11—illustrates a specimen of a floral receptacle showing the arrangement of the pistils (stamens removed); FIG. 12—illustrates a specimen of a flowering stem; FIG. 13—illustrates a specimen of a main branch; FIG. 14—illustrates specimens of two leaves with three leaflets with the upper surface being shown at the right and the under surface being shown at the left; FIG. 15—illustrates specimens of two leaves with five leaflets with the upper surface being shown at the left and the under surface being shown at the right; and FIG. 16—illustrates specimens of two leaves with seven leaflets with the upper surface being shown at the top and the under surface being shown at the bottom.

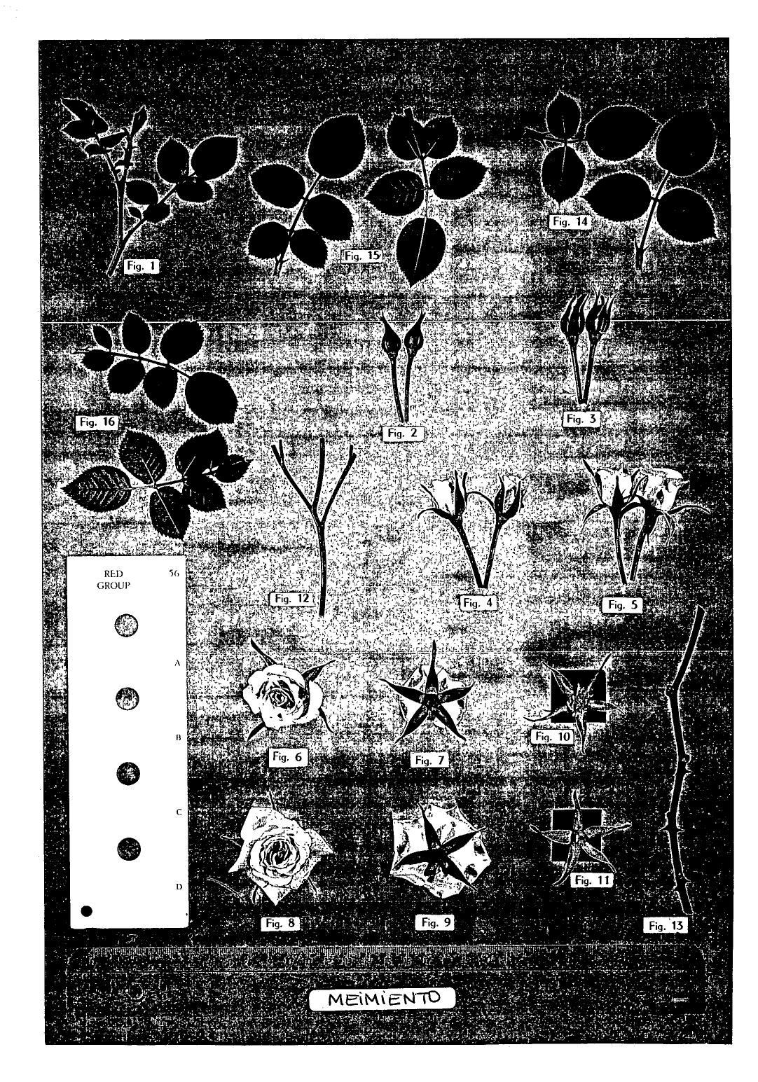

### DETAILED DESCRIPTION

The chart used in the identification of the colors is that of The Royal Horticultural Society (R.H.S. Colour Chart). The terminology which precedes reference to the chart has been added to indicate the corresponding colors in more common terms. The description is based on the observation of two year-old specimens of the new variety which were observed during March while growing on Rosa indica understock in greenhouses at LeCannet des Maures, Var, France.

CLASS: Dwarf Miniature.

PLANT: height—approximately 30 to 35 cm. on average at the end of the growing season.

habit.—bushy.

BRANCHES: color—young stems: Yellow-Green Group 146 D. adult wood: Yellow-Green Group 146 C.

thorns.—size: medium. quantity: sparse as illustrated. color: reddish on young stems and adult wood. configuration: fairly straight on the upper surface and slightly concave on the under surface with a wide and oval base.

LEAVES: petioles.—upper surface: fairly glandular. under surface: rarely bear some small thorns.

stipules.—adnate, pectinate, narrow and pectinate.

leaflets.—number: 3, 5, 7 (most often), and 9. shape: oval. texture: fairly consistent. serration: regular. general appearance: decorative dark green foliage. color (young foliage): upper surface: Yellow-Green Group 147 B. under surface: Yellow-Green Group 147 B suffused with reddish coloration. color (adult foliage): upper surface: Yellow-Green Group 147 A. under surface: Green Group 137 B.

### INFLORESCENCE

number of flowers.—commonly approximately 6 to 15 per stem.

peduncle.—long and rigid, and the length is approximately 4 to 5 cm. on average.

sepals.—upper surface: tomentose. under surface: medium green in coloration.

buds.—shape: elongated. length: approximately 2.5 cm. on average. size: medium for class. color upon opening: upper surface: Red Group 56 D and broadly suffused with Red Group 56 A, 56 B, and 56 C. under surface: Red Group 49 D and suffused with Red Group 49 C.

flower.—form: elongated, double, and cup-shaped. diameter: approximately 6 cm. on average when fully open. color (when opening begins): upper surface: Red Group 56 C suffused with Red Group 56 A. under surface: Red Group 49 D suffused with Red Group 49 C. color (when blooming): upper surface: Red Group 56 C suffused with Red Group 56 A. under surface: Red Group 49 D suffused with Red Group 49 C. color (at end of opening): upper surface: Red Group 56 C suffused with Red Group 56 A. under surface: Red Group 49 D suffused with Red Group 49 C. anthers: yellowish in coloration. stamen number: approximately 75 on average. filaments: whitish in coloration. pistil number: approximately 38 on average. stigmas: red in coloration. styles: whitish in coloration. receptacle: greenish in coloration, and in longitudinal section in the shape of a funnel. lasting qualit : long on the plant.

### DEVELOPMENT

vegetation.—strong.

blooming.—very abundant.

aptitude to bear fruit.—slight.

resistance to diseases.—very good under greenhouse growing conditions.

I claim:

1. A new and distinct variety of Miniature rose plant characterized by the following combination of characteristics:
(a) exhibits a low compact and bushy growth habit,
(b) forms elongated buds that commonly open slowly,
(c) forms in abundance attractive double light pink blossoms,
(d) forms decorative dark green foliage, and
(e) exhibits very good resistance to diseases under greenhouse growing conditions;
substantially as herein shown and described.
